(19) United States
(12) Patent Application Publication
(10) Pub. No.: US 2023/0298765 A1
(43) Pub. Date: Sep. 21, 2023

(54) COMPUTER-IMPLEMENTED PROCESS FOR PREDICTING HEALTH RISK IN REAL-TIME

(71) Applicant: Health Advocate Solutions, Inc., Plymouth Meeting, PA (US)

(72) Inventors: Antonio Legorreta, New Hope, PA (US); Mayur Jigar Patel, Woodland Hills, CA (US); Janelle Sophia Lewis, Boise, ID (US); Thomas Wiese, Saint James, NY (US)

(73) Assignee: Health Advocate Solutions, Inc., Plymouth Meeting, PA (US)

(21) Appl. No.: 17/696,743

(22) Filed: Mar. 16, 2022

(51) Int. Cl. G16H 50/70; G16H 10/60; G16H 50/30; G06F 16/35; G06F 16/335

(52) U.S. Cl. CPC G16H 50/70; G16H 10/60; G16H 50/30; G06F 16/35; G06F 16/335

(57) ABSTRACT

A computer-implemented process receives unstructured patient data pertaining to a patient. Furthermore, the computer-implemented process eliminates redundant words from the unstructured patient data. Additionally, the computer-implemented process generates a numerical representation of the unstructured patient data and a context-specific textual representation of the unstructured patient data. The computer-implemented process classifies, via the machine learning engine, one or more potential portions of the context-specific textual representation of the unstructured data in a risk stratification category. Additionally, the computer-implemented process determines that one or more potential corresponding portions of the numerical representation is also classified in the risk stratification category. Finally, the computer-implemented process outputs, in real-time, an enhancement to the risk stratification category based on the numerical representation also being classified in the risk stratification category.

### BACKGROUND

### 1. Field

This disclosure generally relates to the field of computer-implemented predictive modelling. More particularly, the disclosure relates to computer-implemented predictive modelling for assessing health risk.

### 2. General Background

Even with advances in healthcare services and products, health-related issues are still quite prevalent in the U.S. For example, the Centers for Disease Control and Prevention recently reported in its National Diabetes Statistics Report that over one in ten Americans have diabetes, and approximately one in three American adults have prediabetes. Ostensibly, increased usage of technology-based devices has led to a more sedentary lifestyle for many people, thereby allowing for high rates of obesity, cardiovascular disease, and hypertension, amongst a myriad of other harmful diseases and health-related conditions. Additionally, most people's diets are too focused on excess fat and sugar, rather than proper nutrition, thereby leading to additional health-related issues.

Various conventional technology-based systems attempt to assess the health risk of an individual by aggregating health conditions pertaining to that individual from previous medical claims data. This approach is deficient in its ability to accurately predict the individual's health risk for two main reasons. Firstly, a health-related condition may not be evident in the previous medical claims data. In other words, a previous medical claim is possibly, but not necessarily, an indicator of the same, or related, future medical condition. A patient may have a future medical condition that is completely unrelated to any previous medical condition. Secondly, even when a health-related condition that is pertinent is found in the medical claims data, a significant time delay (e.g., six months in between a service and availability of the data) typically restricts the availability of such findings in a meaningful way. (Such delays may result from coding and/or submission delays for billing purposes, processing, rejection, and revision delays for payment purposes, and storage and transfer delays for data warehousing purposes.)

Accordingly, conventional technology-based systems impose time lags that impair the ability to generate a meaningful predictive assessment as to the health condition of a patient; a patient's health status can significantly change within a time period such as six months. As a result, such conventional systems do not provide health assessments with a sufficient level of accuracy to be relied on in a meaningful way by healthcare practitioners.

### SUMMARY

In one embodiment, a computer-implemented process receives unstructured patient data pertaining to a patient. Furthermore, the computer-implemented process eliminates redundant words from the unstructured patient data. Additionally, the computer-implemented process generates a numerical representation of the unstructured patient data and a context-specific textual representation of the unstructured patient data. The computer-implemented process classifies, via the machine learning engine, one or more potential portions of the context-specific textual representation of the unstructured data in a risk stratification category. Additionally, the computer-implemented process determines that one or more potential corresponding portions of the numerical representation is also classified in the risk stratification category. Finally, the computer-implemented process outputs, in real-time, an enhancement to the risk stratification category based on the numerical representation also being classified in the risk stratification category.

Alternatively, a computer program product may have a computer readable storage device with a computer readable program stored thereon that implements the functionality of the aforementioned processes. As yet another alternative, a system may implement the processes via various componentry.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1 illustrates a computer-implemented process that enhances the accuracy and decreases the time lag in risk stratification through real-time data mining via a Natural Language Processing (“NLP”) configuration.

FIG. 2 illustrates the operation of the data acquisition process component.

FIG. 3 illustrates a feedback loop configuration for the classification model.

FIG. 4A illustrates an example of an enhanced Charlson Comorbidity Index (“CCI”) output generated.

FIG. 4B illustrates an updated CCI based upon the real-time update illustrated in FIG. 4A.

FIG. 5 illustrates a cloud-based computing architecture that may be utilized to implement the NLP configuration illustrated in FIG. 1.

FIG. 6 illustrates a system configuration for the cloud-based computing architecture illustrated in FIG. 5.

FIG. 7 illustrates a computer-implemented process for translating unstructured data into one or more risk stratification categories and predicting one or more health risks of a patient for the one or more risk stratification categories.

### DETAILED DESCRIPTION

A computer-implemented process is provided to predict an individual's health risks in real-time. In contrast with previous systems which had significant lags and relied on known medical conditions, the computer-implemented process is able to provide a real-time assessment based on newly detected and previously unknown health conditions. An enhanced risk stratification approach utilizes an artificial intelligence (AI“)” based system to, firstly, analyze patient interaction data between the patient and clinical personnel, and, secondly, analyze administrative data (e.g., range of claims, known family members, and demographic information). As opposed to using coded and formatted claim data, which introduces the aforementioned time lags, the computer-implemented process receives unstructured patient interaction data as an input. With such unstructured data, the computer-implemented process is able to avoid time lags and generate risk assessments in real-time. Accordingly, health care providers may use the computer-implemented process as a comprehensive risk stratification approach to proactively and preemptively identify the highest risk patients in a given community (e.g., company, city, town, state, country, etc.) to address health risks before the onset of particular health conditions or before treatment options are no longer tenable.

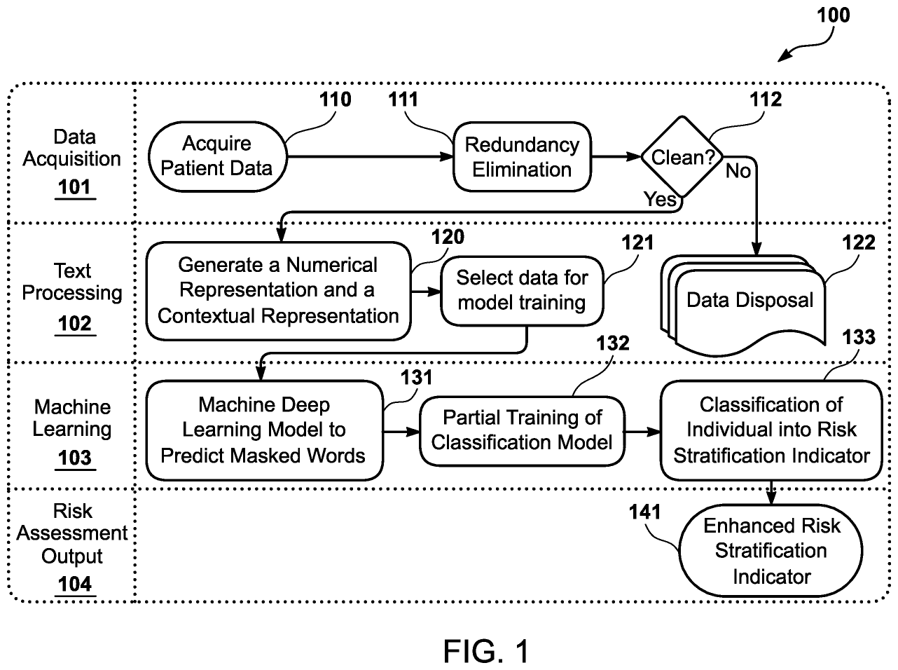

In one embodiment, the computer-implemented process draws upon an extensive database of unstructured patient interaction data. (The unstructured patient interaction data may encompass a variety of data such as clinical conversations, healthcare provider locator requests, claim adjudication issue resolution, and wellness advice.) Given that such data is unstructured, it is inherently difficult to utilize—especially in real-time—without advanced analytical techniques. For example, a data set for a given community could easily have millions of different patient interaction data notes that cannot be analyzed from a practical perspective via conventional systems. Accordingly, the computer-implemented process enhances the accuracy and decreases the time lag in risk stratification through real-time data mining via an NLP configuration 100, as illustrated in FIG. 1.

In particular, the NLP configuration 100 has various components, as illustrated in FIG. 1, that allow for the computer-implemented process to implement AI to provide a practical solution for real-time modelling and predictive analysis of health risks to a patient. The NLP configuration 100 components include the following: (1) a data acquisition component 101; (2) a text processing component 102; (3) a machine learning component 103; and (4) a risk assessment output component 104. Notably, the NLP configuration 100 does not rely on a simple text search of unstructured patient interaction data, which could lead to inaccurate assessments for solving this complex problem; rather, it utilizes context-specific detection of diagnostic indicators that intelligently comprehends the context of a particular reference to a diagnosis.

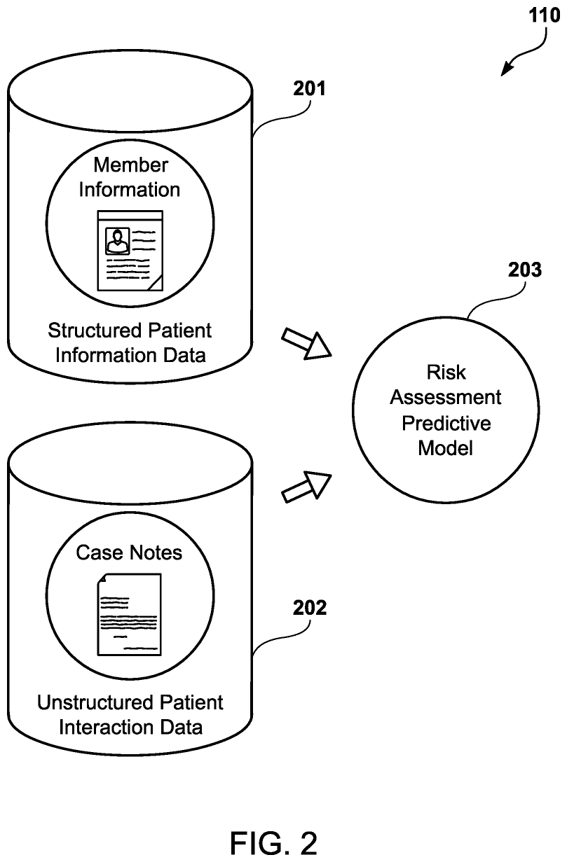

The data acquisition component 101 includes a process component 110 that acquires the patient assessment data, in real-time, pertinent to risk assessment for a particular patient. Turning to FIG. 2, the operation of the process component 110 is illustrated. In particular, the process component 110 ingests data from two databases: (1) an unstructured patient interaction database 202 and (2) a structured patient information database 201. Whereas the unstructured patient interaction database 202 contains data such as case notes (i.e., direct patient quotations, third person entry of the patient's concerns and/or request, and other patient interaction notes), the structured information database 201 includes data such as the names, genders, dates of birth, and familial relationships of the patient's known family members as well as demographic information of the patient. It is these data sets that may be used in conjunction, or individually, to develop the predictive model 203.

Returning to FIG. 1, the a data acquisition component 101 has a redundancy elimination component 111 that is utilized to eliminate redundancies such as common words from the predictive model 203. Additionally, null values may be eliminated by the redundancy elimination component 111. At a decision block 112, the redundancy elimination component 111 determines whether or not redundancies and/or null values have been eliminated. If yes, the NLP configuration 100 advances to a process block 120 of the text processing component 102. At the process block 120, the NLP configuration 100 may generate both a numerical representation and a contextual representation of each word encapsulated in the predictive model 203 (i.e., words from the an unstructured patient interaction database 202 and/or the structured patient information database 201). The numerical representation is utilized for one or more mathematical models, whereas the contextual representation is a text entry that is parsed into individual words called tokens. In one embodiment, the text entry is first converted to its lower-case representation before the aforesaid parsing. Subsequently, the NLP configuration 100 advances to a process block 121 of the text processing component 102 to select data for model training.

If, at the decision block 112, the redundancy elimination component 111 was unable to eliminate redundancies and/or null values, the NLP configuration 100 advances to a process block 122 of the text processing component 102 to dispose of the data. In other words, if redundancies and/or null values cannot be eliminated, the NLP configuration 100 determines that the data is not adequate for further processing.

Returning to the process block 121, the NLP configuration 100 classifies data as a numerical representation or a textual representation, and classifies each independently of each other using different models. For example, a textual representation is classified using a regular expression classifier, whereas a numerical representation is classified using an ensemble machine learning classifier. In one embodiment, each textual representation of the text is searched using the regular expression classifier of each of the health conditions of interest. Furthermore, in one embodiment, each numerical representation of the text is scaled using a trained standard scaler, and a probability for each numerical representation is inferred using the ensemble machine learning classifier. For instance, the numerical representation may be classified as belong to a health condition by the ensemble machine learning classifier if the corresponding probability is greater than a predetermined probability threshold. As an example, which is not intended to be limiting, the predetermined probability threshold is seventy-five percent.

Finally, only the textual representation and numerical representation pairs that are classified by both classifiers (i.e., the regular expression classifier and the ensemble machine learning classifier) as belonging to the same health conditions are considered as the classification of the health condition. The NLP configuration 100 makes the aforesaid determination at the machine learning process block 103, and particularly at process block 131 encapsulated therein that utilizes a machine deep learning model to predict masked words. At the process block 131, the NLP configuration 100, for each text term, determines the probability of the that text term belong to each of the pre-established health conditions by a machine learning ensemble model using the numerical representations of that term; independently, each text term is scanned using a predefined list of regular expressions defined for each of the health conditions. A term which is detected by the regular expression as belong to one of the health conditions and is inferred as belong to that same health condition by the machine learning ensemble model with a probability that exceeds the predetermined probability threshold is categorized as belonging to that health condition.

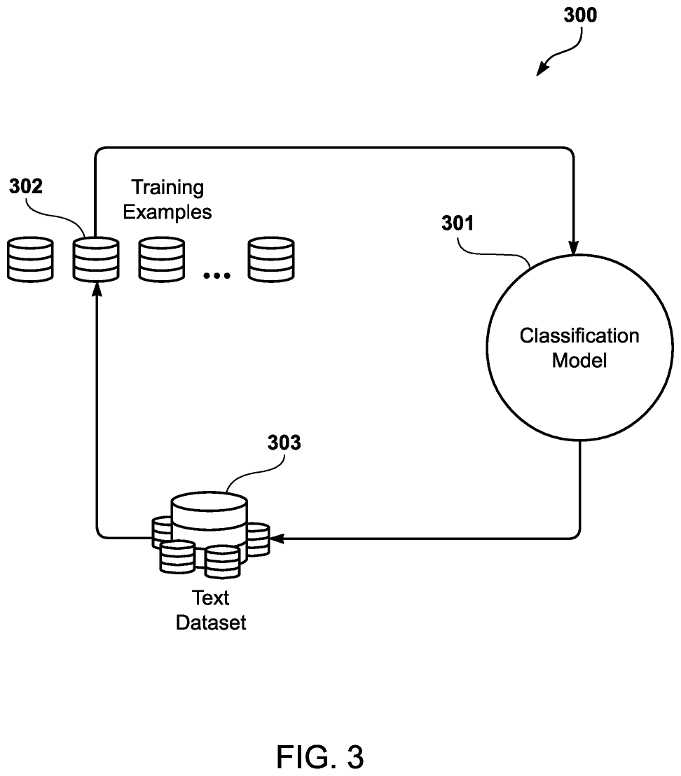

Subsequently, at the process block 132, the NLP configuration 100 partially trains the classifier model by selecting potential examples from which to learn, and the classifier model may then automatically self-learn how to select further potential examples in the future for future learning. In essence, the classifier model needs to be trained to understand which textual occurrences indicate a comorbidity, or other indicator, as opposed to those that do not. For example, the following is a sample of text: “Patient felt pain in her back and a sharp pain in her neck, but now there is no pain. I will wait to hear back from her about how her back is feeling.” The first and third instances of “back” are found to have a high similarity score, whereas the second instance has a low score relative to the other two because it is used as a homonym. Additionally, the first and second instances of “pain” are found to have a high similarity score, whereas the third instance has a low similarity score relative to the first two. FIG. 3 illustrates a feedback loop configuration 300 for the classification model 301. Various training examples 302 are provided from a text dataset 303 to teach the classification model how to select potential examples within much larger data set. This automatic self-learning aspect of the classification model improves computing efficiency because it eliminates allocation of computing resources required to train the classification model in each instance; partial training allows the classification model to train itself, thereby improving computing efficiency.

In one embodiment, subsequent to the foregoing training, returning to FIG. 1, the NLP configuration 100 advances to a process block 132 of the machine learning process block 103 to classify an individual into a predicted risk stratification indicator. (In some embodiments, classification of such individual is not a requirement.) The NLP configuration 100 may segment a sentence into sections, and connect the sections to appropriate person in the text. The following example sentenced shall be used for reference: “John called to discuss Jane's diabetes medication, which he wants to refill.” In this example, the NLP configuration 100 segments the sentence into “John called to discuss . . . which he wants to refill” (segment one) and “Jane's diabetes medication” (segment two). It is clear that segment one is associated with John and segment two is associated with Jane. As a result, the NLP configuration 100 may determine that “diabetes” is associated with Jane.

To accomplish the foregoing segmentation, in one embodiment, the NLP configuration 100 implements a scanning process using the regular expression classifier for the following detection points: (1) common first names; (2) first names associated with the particular matter (e.g., patient's name, caller's name, names of the patient's family members and dependents); (3) common singular and plural family titles (e.g., mother, father, etc.; (4) patient-related titles such as “patient”; (5) age described persons (e.g., “forty year old with diabetes”); and (6) common English pronouns. Initially, the detection of each of the foregoing is considered as separate and individual discussed persons. The sentence is segmented at each detection point, and the sentence segments are associated with the consolidate person preceding it. Similarly, the detected condition in the sentence section is associated with the same person. Of particular note is that the person may be the patient associated with the health-related matter, the patient's family member or dependent, or a third party person who is not connected to the patient in any administrative manner. Also, such association of the detected health condition with the appropriate person may or may not have an impact on adjusting the risk stratification.

Subsequently, the NLP configuration 100 combines the detected sentence portions which refer to the same person. The NLP configuration 100 assumes that name detections at the first and second detection points are names associated with the same person based on each unique name corresponding to a unique person. At the third detection point, the known family titles of the patient's family members and dependents are used to associate the family member with the singular family title detection. The family members' or dependents' family titles may be derived from either known records or separate health-related matter notes, such as caller name and relation. At the fifth detection point, age detections with the same age are associated with the same person under the assumption that each unique age is a unique person. The same age detections are then associated with the patient or the family members or dependents, if the date of birth and date of matter note match the age detection. At the fourth detection point, all detections or patient related titles are associated with the patient associated with the patient corresponding to the matter note.

Finally, using the pronoun detections at the sixth detection point, the NLP configuration 100 associates each pronoun with the persons consolidated up to this point as follows. Firstly, the NLP configuration 100 associates singular male pronouns with the closest preceding consolidated male person in the text, and the NLP configuration 100 associates singular female pronouns with the closest preceding consolidated female person in the text. Secondly, the NLP configuration 100 associates plural pronouns with either of the two preceding it: (1) a plural consolidated person such as “parents”; or (2) two separate consolidated singular persons. As a result, an individual may be classified into a predicted risk stratification indicator.

Returning to FIG. 1, the NLP configuration 100, after performing machine learning at the process block 103, advances to a process block 104 to generate an enhanced risk stratification output. In particular, at a process block 140, the NLP configuration 100 performs an assignment of a risk stratification assessment via an enhanced risk stratification indicator 141. As an example of such a risk stratification assessment, the CCI, which is a method for grouping comorbidities based on the International Classification of Diseases (“ICD”) diagnosis codes. The NLP configuration 100 may create a binary comorbidity indicator for each of the seventeen comorbidity groups of the CCI; or another binary risk stratification indicator. The binary aspect includes both the patient interaction data and the patient information data. The regular expression classifier and the ensemble classifier are trained by the NLP configuration 100 via a term list for each health condition of interest. Using the descriptions of the ICD codes associated with each CCI comorbidity, or health condition, a list of terms indicated the corresponding comorbidity is generated. The NLP configuration 100 trains the regular expression classifier and the ensemble classifier to detect and classify the seventeen CCI comorbidities in matter notes. Subsequently, in one embodiment, the detected comorbidities are then associated with an individual. Finally, the comorbidity indicator, or other risk stratification indicator, is configured to enhance the CCI, or other risk stratification tool. CCI classification is just an example a risk stratification indicator—other risk stratification approaches may be utilized in addition or in the alternative.

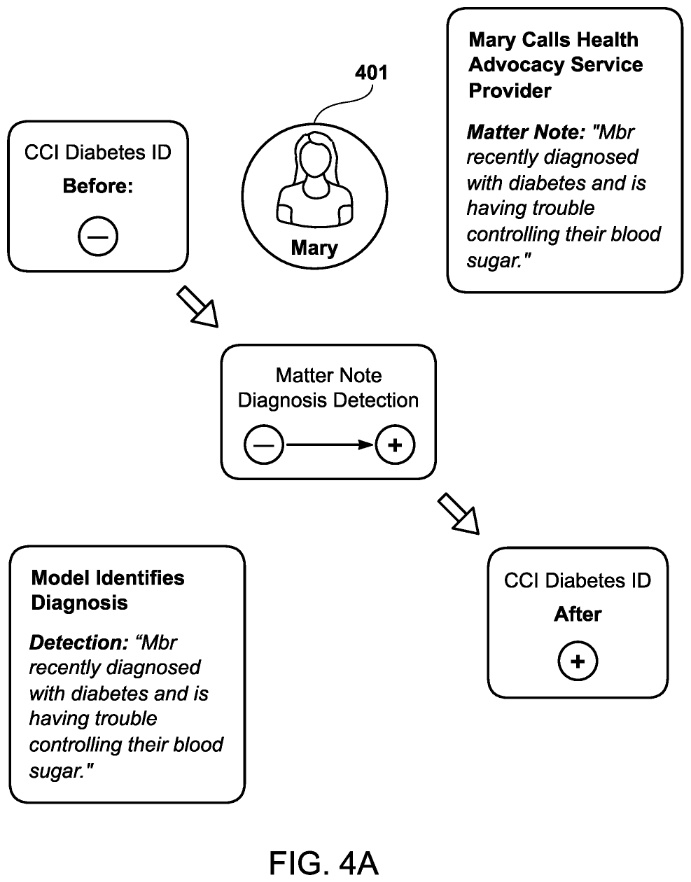

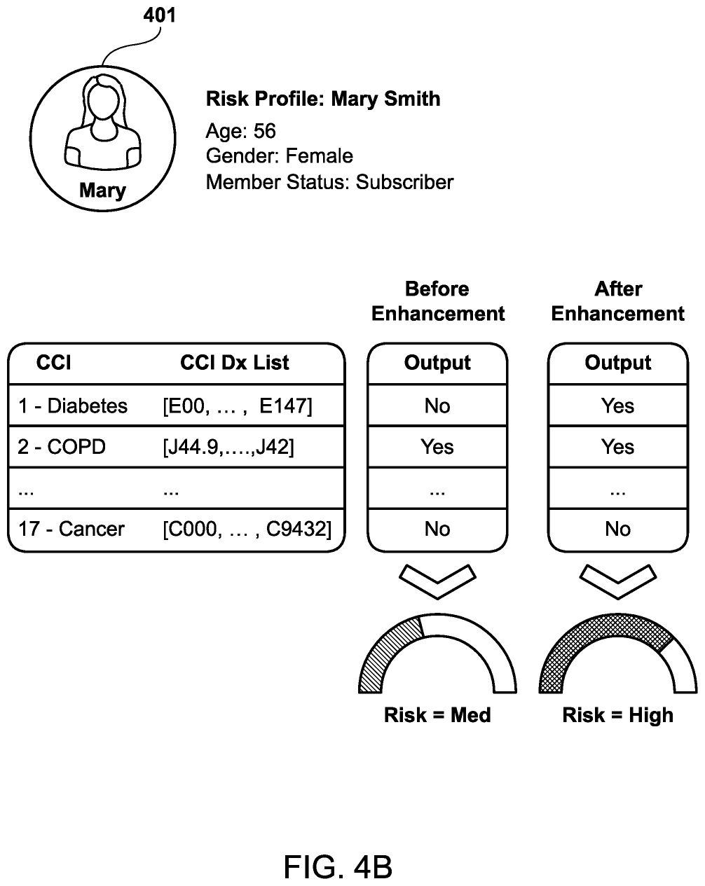

FIG. 4A illustrates an example of an enhanced CCI output generated. In this example, a patient 401 named “Mary” calls a health advocacy service provider. The prior CCI indicator for diabetes was negative. During the phone call, a matter note was generated to indicate that the patient was recently diagnosed with diabetes. In real-time based on the matter note, the NLP configuration 100 enhances the CCI indicator to indicate a positive outcome for the patient 401.

FIG. 4B illustrates an updated CCI based upon the real-time update illustrated in FIG. 4A. After the real-time enhancement, the CCI indicator risk level is increased form medium to high. As a result, a patient risk profile may monitored and updated in real-time to preemptively address health-related issues. In one embodiment, a risk score is calculated; while in other embodiments, a risk assessment may be performed without a particular risk score.

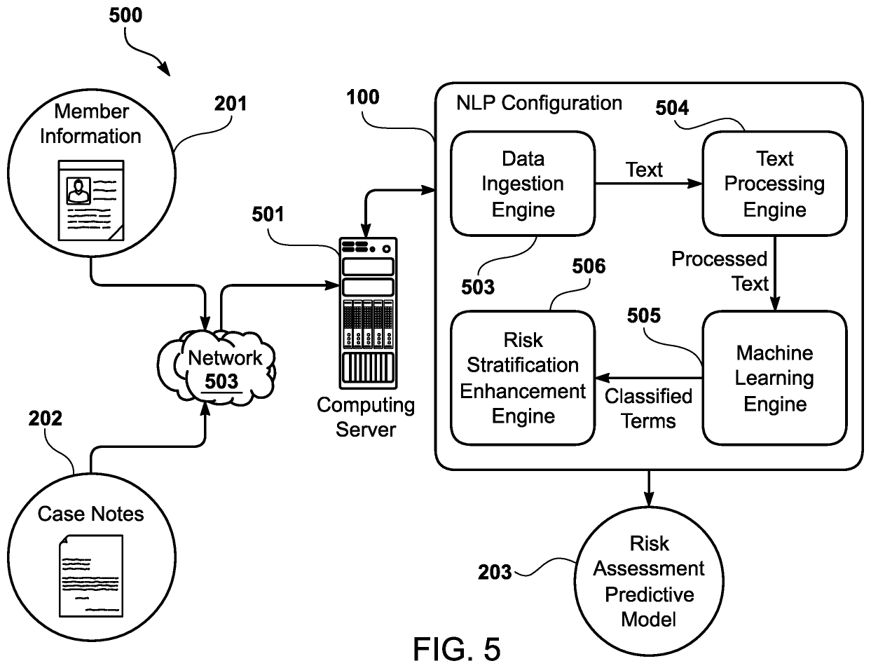

FIG. 5 illustrates a cloud-based computing architecture 500 that may be utilized to implement the NLP configuration 100 illustrated in FIG. 1. In particular, a computing server 501 receives, via a computerized network 502, the unstructured patient interaction data from the unstructured patient interaction database 202 and the structured patient information data from the structured patient information database 201. The computing server 501 is in operable communication with the NLP configuration 100, which has a data ingestion engine 503 that ingests the unstructured patient interaction data and the structured patient information data. Accordingly, the data ingestion engine 503 implements the data acquisition component 101 illustrated in FIG. 1. Furthermore, the data ingestion engine 503 provides the unstructured patient interaction data and the structured patient information data to a text processing engine 504, which implements the text processing component 102 illustrated in FIG. 1. After processing the textual data, the text processing engine 504 provides the processed textual data to a machine learning engine 505, which implements the machine learning component 103 illustrated in FIG. 1. Finally, after classifying terms according to a given risk stratification tool, the machine learning engine 505 sends the classification to a risk stratification enhancement engine 505, which implements the risk assessment output component 104 of FIG. 1. As a result, the NLP configuration 100 of the cloud-based computing architecture 500 generates, in real-time, the risk assessment predictive model 203 that may be used to predict health risks for a given patient and/or a community at large.

In one embodiment, the NLP configuration 100 processes numerical representation and contextual representation pairs solely from the unstructured patient data, and then adjusts the risk assessment output, prior to outputting, based on the structured patient data. In another embodiment, the NLP configuration 100 processes numerical representation and contextual representation pairs both from the unstructured patient data and the structured patient data from the outset.

The use of the phrase “real-time” is intended herein to be measured from the time from which data is received by the computing server to the time in which a risk stratification indicator is enhanced via the risk assessment predictive model 203, and is intended to connote a delay that is relatively imperceptible to a human.

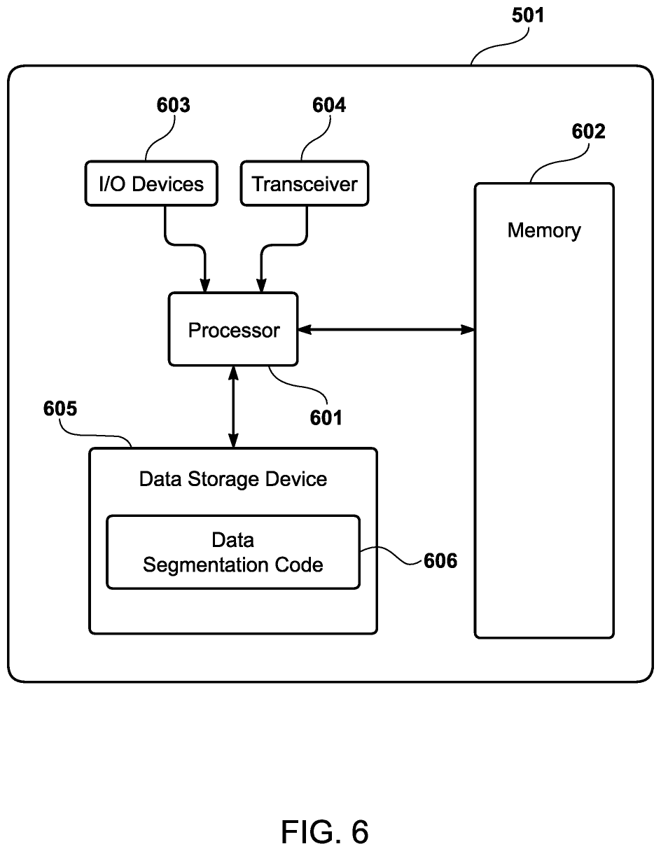

FIG. 6 illustrates a system configuration for the cloud-based computing architecture 500 illustrated in FIG. 5. A processor 601 may be specialized for NLP operations, namely text processing to eliminate redundancies to improve computing efficiency to allow real-time risk stratification enhancement output and on-the-fly machine learning to allow for real-time classification. The system configuration may also include a memory device 602, which may temporarily store a segmented pair data structure that delineates between the pair of textual representation data and numerical representation data, thereby preserving the computing independence of the two different types of data for application of the different classifier models. The segmented pair data structure may one of a variety of data structures, such as an array, linked list, tree, or the like. The segmented data structure improves the operation of the computing server 501 (illustrated in FIG. 5) by allowing each classifier model to only have to search the pertinent data set, thereby reducing computing time and allowing for real-time risk stratification assessment. Furthermore, the memory device 602 may temporarily store computer readable instructions performed by the processor 601. As an example of such computer readable instructions, a data storage device 605 within the system configuration may store data segmentation code 606 that may be utilized to compose, maintain, and access the data segmentation data structure. Various devices (e.g., keyboard, microphone, mouse, pointing device, hand controller, joystick, display screen, holographic projector, etc.) may be utilized for input/output (“I/O”) devices 603. The system configuration may also have a transceiver 604 to send and receive data. Alternatively, a separate transmitter and receiver may be used instead.

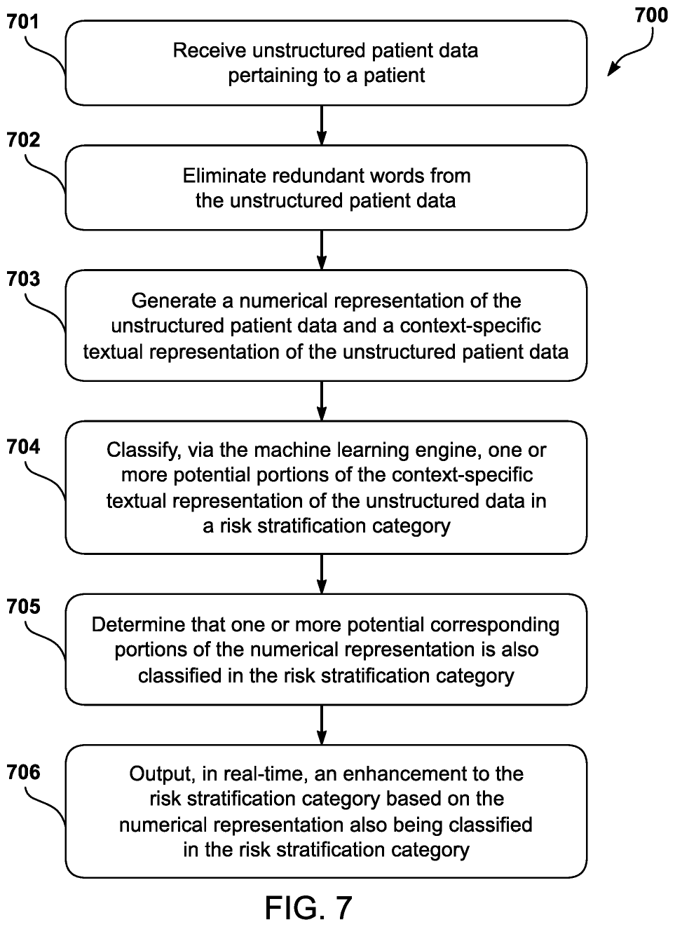

FIG. 7 illustrates a computer-implemented process 700 for translating unstructured data into one or more risk stratification categories and predicting one or more health risks of a patient for the one or more risk stratification categories. At a process block 701, the computer-implemented process 700 receives unstructured patient data pertaining to a patient. Furthermore, at a process block 702, the computer-implemented process 700 eliminates redundant words from the unstructured patient data. Additionally, at a process block 703, the computer-implemented process 700 generates a numerical representation of the unstructured patient data and a context-specific textual representation of the unstructured patient data. At a process block 704, the computer-implemented process 700 classifies, via the machine learning engine, one or more potential portions of the context-specific textual representation of the unstructured data in a risk stratification category. Additionally, at a process block 705, the computer-implemented process 700 determines that one or more potential corresponding portions of the numerical representation is also classified in the risk stratification category. Finally, at a process block 706, the computer-implemented process 700 outputs, in real-time, an enhancement to the risk stratification category based on the numerical representation also being classified in the risk stratification category.

It is understood that the apparatuses, systems, computer program products, and processes described herein may also be applied in other types of apparatuses, systems, computer program products, and processes. Those skilled in the art will appreciate that the various adaptations and modifications of the embodiments of the apparatuses, systems, computer program products, and processes described herein may be configured without departing from the scope and spirit of the present apparatuses, systems, computer program products, and processes. Therefore, it is to be understood that, within the scope of the appended claims, the present apparatuses, systems, computer program products, and processes may be practiced other than as specifically described herein.

We claim:

1. A computer-implemented process comprising:
means for receiving unstructured patient data pertaining to a patient;
means for eliminating redundant words from the unstructured patient data;
means for generating a numerical representation of the unstructured patient data and a context-specific textual representation of the unstructured patient data;
means for classifying, via the machine learning engine, one or more potential portions of the context-specific textual representation of the unstructured data in a risk stratification category;
means for determining that one or more potential corresponding portions of the numerical representation is also classified in the risk stratification category; and
means for outputting, in real-time, an enhancement to the risk stratification category based on the numerical representation also being classified in the risk stratification category.

2. The computer-implemented process of claim 1, wherein the means for determining that that the one or more potential corresponding portions of the numerical representation is also classified in the risk stratification category determines that probability threshold associated with a probability that the one or more potential corresponding portions belong to one or more health conditions associated with the risk stratification category is exceeded.

3. The computer-implemented process of claim 1, further comprising means for partially training the machine learning engine via one or more training data sets to automatically self-learn to select the one or more potential portions.

4. The computer-implemented process of claim 1, further comprising means for receiving structured patient data.

5. The computer-implemented process of claim 4, wherein the means for outputting, in real-time, the enhancement to the risk stratification category utilizes one or more portions of the structured patient data to adjust the enhancement prior to performing the outputting.

6. The computer-implemented process of claim 1, further comprising means for scanning one or more sentences the context-specific textual representation for one or more detection points and segmenting the context-specific textual representation at the one or more detection points.

7. The computer-implemented process of claim 6, wherein the one or more detection points are selected from the group consisting of: common first names, first names associated with a particular matter, common singular and plural family titles, patient-related titles, age described persons, and common English pronouns.

8. A computer program product comprising a computer readable storage device having a computer readable program stored thereon, wherein the computer readable program when executed on a computer causes the computer to:
receive unstructured patient data pertaining to a patient;
eliminate redundant words from the unstructured patient data;
generate a numerical representation of the unstructured patient data and a context-specific textual representation of the unstructured patient data;
classify, via the machine learning engine, one or more potential portions of the context-specific textual representation of the unstructured data in a risk stratification category;
determine that one or more potential corresponding portions of the numerical representation is also classified in the risk stratification category; and
output, in real-time, an enhancement to the risk stratification category based on the numerical representation also being classified in the risk stratification category.

9. The computer program product of claim 8, wherein the computer is further caused to determine that a probability threshold associated with a probability that the one or more potential corresponding portions belong to one or more health conditions associated with the risk stratification category is exceeded.

10. The computer program product of claim 8, wherein the computer is further caused to partially training the machine learning engine via one or more training data sets to automatically self-learn to select the one or more potential portions.

11. The computer program product of claim 8, wherein the computer is further caused to receive structured patient data.

12. The computer program product of claim 11, wherein computer is further caused to utilize one or more portions of the structured patient data to adjust the enhancement prior to performing the outputting.

13. The computer program product of claim 8, wherein the computer is further caused to scan one or more sentences the context-specific textual representation for one or more detection points and segmenting the context-specific textual representation at the one or more detection points.

14. The computer program product of claim 13, wherein the one or more detection points are selected from the group consisting of: common first names, first names associated with a particular matter, common singular and plural family titles, patient-related titles, age described persons, and common English pronouns.

15. A computer-implemented system comprising:
an unstructured patient database that stores unstructured patient data pertaining to a patient; and
a computing server comprising a processor configured to perform the following:
receive unstructured patient data pertaining to a patient,
eliminate redundant words from the unstructured patient data,
generate a numerical representation of the unstructured patient data and a context-specific textual representation of the unstructured patient data,
classify, via the machine learning engine, one or more potential portions of the context-specific textual representation of the unstructured data in a risk stratification category,
determine that one or more potential corresponding portions of the numerical representation is also classified in the risk stratification category, and
output, in real-time, an enhancement to the risk stratification category based on the numerical representation also being classified in the risk stratification category.

16. The computer-implemented system of claim 15, wherein the processor is further configured to determine that a probability threshold associated with a probability that the one or more potential corresponding portions belong to one or more health conditions associated with the risk stratification category is exceeded.

17. The computer-implemented system of claim 15, wherein the processor is further configured to partially train the machine learning engine via one or more training data sets to automatically self-learn to select the one or more potential portions.

18. The computer-implemented system of claim 15, wherein the processor is further configured to receive structured patient data.

19. The computer-implemented system of claim 18, wherein the processor is further configured to utilize one or more portions of the structured patient data to adjust the enhancement prior to performing the outputting.

20. The computer-implemented system of claim 15, wherein the processor is further configured to scan one or more sentences the context-specific textual representation for one or more detection points and segmenting the context-specific textual representation at the one or more detection points.
